(19) United States
(12) Patent Application Publication
(10) Pub. No.: US 2025/0221634 A1
(43) Pub. Date: Jul. 10, 2025

(54) METHOD FOR CALIBRATING A PLURALITY OF WEARABLE INERTIAL MEASUREMENT UNITS

(71) Applicant: Robert Bosch GmbH, Stuttgart (DE)

(72) Inventors: Dmitry Dzhigaev, Malmö (SE); Aadarsh Kumar Singh, Reutlingen (DE)

(21) Appl. No.: 18/972,001

(22) Filed: Dec. 6, 2024

(30) Foreign Application Priority Data
Jan. 8, 2024 (DE) 10 2024 200 156.2

(51) Int. Cl. A61B 5/11; A61B 5/00

(52) U.S. Cl. CPC A61B 5/1126; A61B 5/0004; A61B 5/0024; A61B 2560/0223; A61B 2562/0219

(57) ABSTRACT

A method for calibrating a plurality of wearable inertial measurement units. The method includes: (A) align a plurality of wearable inertial measurement units with respect to a heading direction; (B) initialize the coordinate systems of the wearable inertial measurement units; (C) Assign each wearable inertial measurement unit to a movable part of a body; (D) Mount each wearable inertial measurement unit to the respectively assigned movable part of the body; (E) Arrange the movable parts of the body in a prescribed manner; (F) Perform a mounted state calibration for each wearable inertial measurement unit.

### CROSS REFERENCE

The present application claims the benefit under 35 U.S.C. § 119 of German Patent Application No. DE 10 2024 200 156.2 filed on Jan. 8, 2024, which is expressly incorporated herein by reference in its entirety.

### FIELD

The present invention relates to the calibration routine of the inertial measurement units for 3D motion capture using a network of interconnected devices and sensor fusion of accelerometers and gyroscopes for orientation estimation.

### BACKGROUND INFORMATION

Accurate determination of a human body pose in 3D space is highly important for applications in the motion capture industry (3D avatars, animation, cinema production), healthcare (rehabilitation, gait analysis), augmented reality (AR), and virtual reality (VR). Having access to affordable, low-power consumption, and wireless systems will greatly promote progress in the abovementioned fields.

This task requires reliable orientation determination of wearable devices in 3D space. This is achieved by employing Inertial Measurement Units (IMUs) as hardware base. The sensor fusion algorithm based on the Kalman filter, combining accelerometer and gyroscope data, provides relative orientation data in the form of Euler angles or quaternions.

Using orientation data has the advantage of high relative orientation precision in 3D space and low power consumption on the order of 3 mA (2.68-3.44 mA, depending on the number of devices). Combined with the star-shaped architecture of the Smart Connected Sensor (SCS) network (see FIG. 1) and the use of Bluetooth low-energy (BLE) connection to the end user, the proposed system provides mobile and accurate quantitative data generation and analysis.

Current drawbacks of mobile motion capture systems are constraints on the mounting orientation of the devices on the body parts and time- or movement-dependent drift of the orientation information when no magnetometer sensor is used. Therefore, calibration of the sensors' orientation and drift compensation routines are required.

The present invention provides a mounting and drift correction routine for the network of SCS wearable devices in 3D motion tracking.

### SUMMARY

The present invention relates to a method for calibrating a plurality of wearable inertial measurement units. According to an example embodiment of the present invention, the method comprises the following steps:
(A) Align a plurality of wearable inertial measurement units with respect to a heading direction;
(B) Initialize the coordinate systems of the wearable inertial measurement units;
(C) Assign each wearable inertial measurement unit to a movable part of a body;
(D) Mount each wearable inertial measurement unit to the respectively assigned movable part of the body;
(E) Arrange the movable parts of the body in a prescribed manner;
(F) Perform a mounted state calibration for each wearable inertial measurement unit.

The present invention provides calibration routines for mounting and drift compensation of IMU orientation in 3D space which is suitable to provide a reliable 3D avatar visualization. Predefined calibration poses allow the remapping of coordinate systems for each device to offer one-to-one 3D avatar visualization and data, which can be directly employed for quantitative data analysis. Further ideas of pose detection and translational movement prediction based on quaternions are disclosed.

The calibration of the SCS mounting is based on the fact that the heading direction of the avatar is defined by the startup procedure, where all devices are aligned with the chosen heading direction. The orientation drift of sensors is corrected when the mounting calibration of the devices is known. This two-step procedure allows to run 3D avatar applications non-stop for extended periods of time.

An example embodiment of the present invention provides that in step A, the plurality of wearable inertial measurement units is aligned by placing them in a common receptacle unit. Advantageously, this mounting device, for example a box, ensures that the IMUs adopt a specified orientation and position in relation to the heading direction.

An example embodiment of the present invention provides that step G is repeated after some time to compensate for a drift of orientation. An advantage of repeating the calibration is that the drift is recognized and compensated for in the future as well. This means that calibration has to be carried out less frequently.

An example embodiment of the present invention provides that the wearable inertial measurement units establish leaf nodes which communicate with a central node wirelessly.

### BRIEF DESCRIPTION OF THE DRAWINGS

FIG. 1 shows a scheme of leaf-central design of a SCS network of IMU devices, according to an example embodiment of the present invention.

FIGS. 2A and 2B show a two-step calibration of heading and drift of a plurality of wearable IMUs, according to an example embodiment of the present invention.

FIG. 3 shows a method for calibrating a plurality of wearable inertial measurement units according to an example embodiment of the present invention.

### DETAILED DESCRIPTION OF EXAMPLE EMBODIMENTS

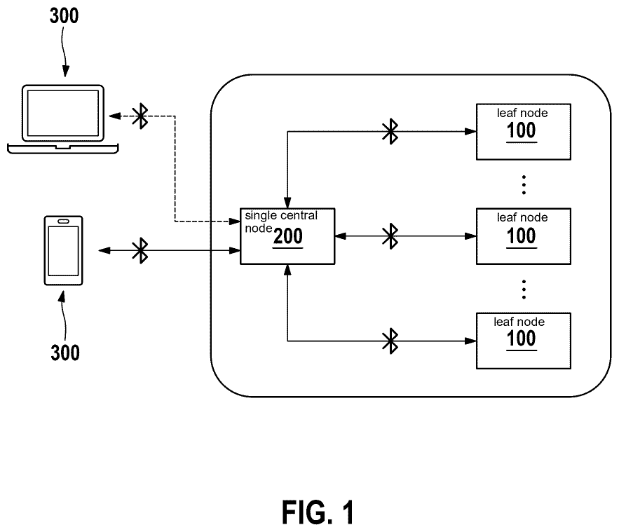

FIG. 1 shows a scheme of leaf-central design of a SCS network of IMU devices. Leaf nodes 100 communicate wirelessly, e.g. via Bluetooth low energy (BLE) with a single central node 200. The latter is connected to a user device 300, e.g. a smart phone, a notebook computer, etc.

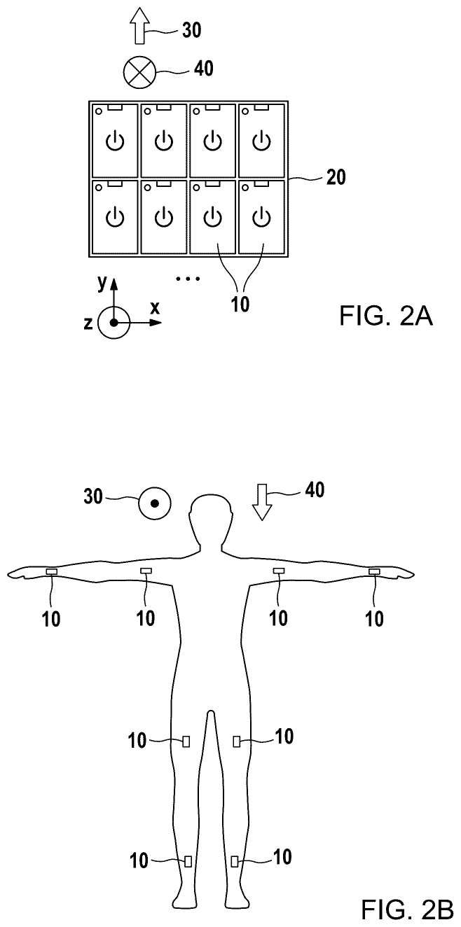

FIGS. 2A and 2B show a two-step calibration of heading and drift of a plurality of wearable IMUs.

FIG. 2A illustrates an initial orientation of a plurality of aligned SCS boards 10 placed in an alignment box 20 which determines heading direction of a 3D avatar. The local coordinate system of the sensors is shown as x, y, z vectors. Gravitation 40 directs into the table top, on which the alignment box is placed.

FIG. 2B illustrates a scheme of a T-pose calibration of mounting of SCS boards 10 on a human body relative to heading. Each wearable inertial measurement unit is assigned and mounted to a movable part of a body, which in the depicted case is a limb of a human.

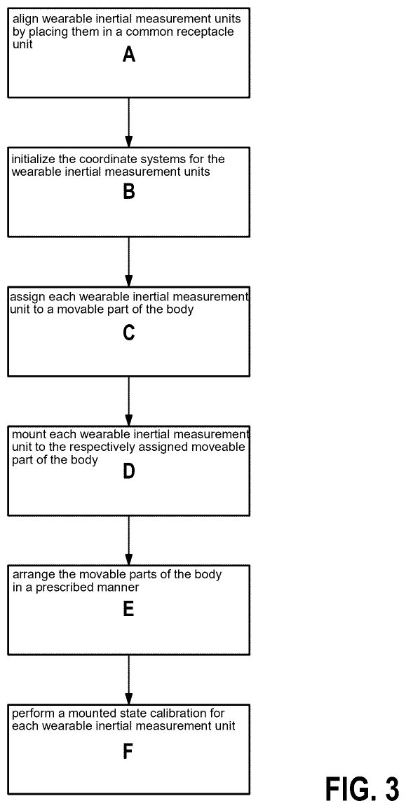

FIG. 3 shows a method for calibrating a plurality of wearable inertial measurement units according to the present invention.

The method comprises the steps of:
(A) Align a plurality of wearable inertial measurement units with respect to a heading direction;
(B) Initialize the coordinate systems of the wearable inertial measurement units;
(C) Assign each wearable inertial measurement unit to a movable part of a body;
(D) Mount each wearable inertial measurement unit to the respectively assigned movable part of the body;
(E) Arrange the movable parts of the body in a prescribed manner;
(F) Perform a mounted state calibration for each wearable inertial measurement unit.

The aim of calibration routines is to compensate for two types of errors:

A first error type is a mounting error, which is a change in orientation of the sensors in the SCS devices when placed in the alignment box lying on the desk versus when worn on the body.

A second error type is a sensor drift error, which is an orientation sensor data error accumulated over time due to the movements of the user wearing the devices for the demo. This error results from integration operation withing the fusion algorithm and noise in the sensor data.

### Setting Up SCS Boards

Choose the direction of the user performing the demo, which stands for heading direction. All the SCS devices must be aligned before the demonstration and coordinate systems are initialized as shown in FIG. 2a.

### Mounting Compensation

The user can assign the body positions to SCS wearables by mapping body positions to the corresponding MAC-ID of the SCS wearable in a config file.

Mount devices on corresponding limbs as set up in the config file.

The user must stand in a T-shape pose in the heading direction as shown in FIG. 2 b, then perform mounting calibration by recording the current orientation of the devices and remapping coordinate systems accordingly.

### Drift Compensation

While performing the demo, the orientation drift occurs due to the integration of gyroscope readouts and noise in IMUs. The user performs the T-pose again in the heading direction. Then, the drift compensation is applied by subtracting the current SCS boards' orientation misalignment with respect to a known calibration pose.

### Formal Description

The orientation of a 3D mesh of the Avatar is defined by the current reading of a game rotation vector sensor (GRV) in the form of a unit quaternion \hat{q}ᵢ. This value is received directly as a virtual sensor GRV from the SCS board and applied as a rotation of a mesh from the original avatar pose.

The update rule for the quaternion to be applied as a rotation of the mesh is the following (implemented in the update method of the Avatar class):

$${\hat{q}}_{i} = {\hat{q}}_{i}^{mounting} ⊗ {\hat{q}}_{i} ⊗ {\hat{q}}_{i}^{drift} \qquad ( 1 )$$

The local rotation of any child mesh connected to the parent mesh is calculated as follows:

$${\hat{q}}_{child}^{local} = {\hat{q}}_{parent}^{*} ⊗ {\hat{q}}_{child}^{global} \qquad ( 2 )$$

here \hat{q}* is a conjugate operation.

Here, \hat{q}ᵢ^{mounting}, \hat{q}ᵢ^{drift} are correction quaternions stored as the amounting adrift Avatar properties and updated via T-pose resets as follows.

After the devices are turned on in the starting box in an aligned manner and mounted on the body with the predefined heading the mounting of the devices is reset as follows:

The person stands in the T-pose [or any other pre-defined pose in 3D view] and a set of current quaternion readouts multiplied by quaternion fixing a drift is recorded: \hat{q}ᵢ^{†}=\hat{q}ᵢ^{drift}⊗\hat{q}ᵢ

The quaternion used to compensate for mounting is calculated as the conjugate of \hat{q}ᵢ^{†}: \hat{q}ᵢ^{mounting}=(\hat{q}ᵢ^{drift}⊗\hat{q}ᵢ)*

If the drift of the GRV sensor readout occurs during the demo operation the T-pose reset for drift is done similarly

The person stands in the T-pose and a set of current quaternion readouts multiplied by quaternion fixing a mounting is recorded: \hat{q}ᵢ^{‡}=\hat{q}ᵢ⊗\hat{q}ᵢ^{mounting}

The quaternion used to compensate for drift is calculated as; the conjugate of \hat{q}ᵢ^{‡}: \hat{q}ᵢ^{drift}=(\hat{q}ᵢ⊗\hat{q}ᵢ^{mounting})

### Simple Pose Detection

Pose detection is possible based on the provided hardware and a mathematical framework. A normalized quaternion \hat{q}_{ref} defines a reference pose. Each device is assigned with a normalized quaternion \hat{q}.

To detect a pose, the following routine is applied:
1. Calculate relative angles between devices as the angular part of the quaternion difference δ\hat{q}=\hat{q}⊗\hat{q}*_{ref}, where δ\hat{q}=a+b\vec{i}+c\vec{j}+d\vec{k}:

$$θ = 2 {cos}^{- 1} ( a ) = 2 {sin}^{- 1} ( \sqrt{{b}^{2} + {c}^{2} + {d}^{2}} ) \qquad ( 3 )$$

2. Create lookup table for j poses p=(p₁, p₂, . . . pⱼ), where pⱼ=(θ₁, θ₂, . . . , θᵢ) and i∈[1, N] is the number of sensors mounted on the human body at predefined locations.
3. The score for a given pose pⱼ can be represented as R and describes how well the current position resembles the pre-recorded one:

$$R = \frac{1}{N} {\sum }_{i = 1}^{N} {( {θ}_{i} - {θ}_{i}^{p} )}^{2} \qquad ( 4 )$$

What is claimed is:

1. A method for calibrating a plurality of wearable inertial measurement units comprising the following steps:
(A) aligning a plurality of wearable inertial measurement units with respect to a heading direction;
(B) initializing coordinate systems of the wearable inertial measurement units;
(C) respectively assigning each of the wearable inertial measurement unit to a movable part of a body;
(D) mounting each wearable inertial measurement unit to the respectively assigned movable part of the body;
(E) arranging the movable parts of the body in a prescribed manner; and
(F) performing a mounted state calibration for each of the wearable inertial measurement units.

2. The method according to claim 1, wherein, in step A, the plurality of wearable inertial measurement units is aligned by placing them in a common receptacle unit.

3. The method according to claim 1, wherein step F is repeated after some time to compensate a drift of orientation.

4. The method according to claim 1, wherein the wearable inertial measurement units establish leaf nodes which communicate with a central node wirelessly.
